(19) United States
(12) Patent Application Publication
(10) Pub. No.: US 2021/0022651 A1
(43) Pub. Date: Jan. 28, 2021

(54) THREE-DIMENSIONAL MICROFLUIDIC ACTUATION AND SENSING WEARABLE DEVICE FOR IN-SITU BIOFLUID PROCESSING AND ANALYSIS

(71) Applicant: The Regents of the University of California, Oakland, CA (US)

(72) Inventors: Sam EMAMINEJAD, Los Angeles, CA (US); Haisong LIN, Los Angeles, CA (US)

(73) Assignee: The Regents of the University of California, Oakland, CA (US)

(21) Appl. No.: 17/040,452

(22) PCT Filed: Mar. 22, 2019
(86) PCT No.: PCT/US2019/023594

§ 371 Date: Sep. 22, 2020

Related U.S. Application Data
(60) Provisional application No. 62/647,320, filed on Mar. 23, 2018.

(51) Int. Cl. A61B 5/1477; A61B 5/145; G06F 1/16

(52) U.S. Cl. CPC A61B 5/1477; G06F 1/163; A61B 5/14517; A61B 5/1451

(57) ABSTRACT

A device for biofluid processing and analysis includes a microfluidic module including multiple stacked layers, each layer of the stacked layers defines a respective conduit, and conduits of the stacked layers are interconnected to provide a flow path for a biofluid.

### CROSS-REFERENCE TO RELATED APPLICATIONS

This application claims the benefit of U.S. Provisional Application No. 62/647,320, filed Mar. 23, 2018, the contents of which are incorporated herein by reference in their entirety.

### TECHNICAL FIELD

This disclosure generally relates to a device for biofluid processing and analysis.

### BACKGROUND

Wearable sensing technologies are poised to transform personalized and precision medicine, as these technologies allow the longitudinal and objective assessment of the health status of individuals in real-time. Comparative non-invasive wearable sensors are capable of tracking physical activities and vital signs, but these sensors generally fail to access molecular-level biomarker information that can provide insight into the body's dynamic chemistry. In that regard, a class of wearable devices are desired to frequently and non-invasively sample, process and analyze biofluids such as sweat and interstitial fluid, which are rich reservoirs of biomarkers. Advances in electrochemical sensor development, flexible device fabrication and integration technology, and low power electronics have set forth a path toward implementing the key functionalities for in-situ tracking of biomarkers. Comparative wearable biomarker sensors demonstrated electrochemical and colorimetric sensing interfaces for on-body detection of analytes in micro- to millimolar ranges. These sensors rely on the analysis of biofluid samples that are passively collected in absorbent pads or two-dimensional (2D) microfluidic housings. Accordingly, their analytical operations are constrained due to the lack of control on biofluid flow and storage.

It is against this background that a need arose to develop the embodiments described herein.

### SUMMARY

Wearable non-invasive biomarker sensors provide a foundation for personalized medicine by providing frequent and real-time measurements of biomarker molecules in autonomously retrieved biofluid samples of individuals. To realize a full range of capabilities of these sensors, compartmentalized sample processing and analysis are desired, fundamentally involving active microfluid handling capabilities. Comparative wearable non-invasive biomarker sensors rely on in-situ analysis of biofluid samples (e.g., sweat and interstitial fluid) that are passively collected in absorbent pads or 2D microfluidic housings. The spatial constraints of these platforms and their lack of active control on biofluids constrain their efficiency, diversity and frequency of end-point assessments. Here, in some embodiments, by including a suite of programmable electro-fluidic interfaces, integrated within a multi-layer flexible microfluidic device, demonstration is made of biofluid manipulation functionalities including pumping, mixing and valving for wearable sample analysis. The device is formed by stacking thin layers of adhesive polymeric substrates, patterned with electrodes (for electro-fluidic flow induction, compartmentalization and electrochemical sensing) and pre-defined microfluidic conduits to form channels, vias, and valves. To achieve autonomous and controllable biofluid actuation and sensing, the microfluidic device is interfaced with a miniaturized and wireless printed circuit board (PCB). The desired biofluid operations on-body is validated through human subject testing. This integrated device merges a technological gap between biofluid sample handling and analysis in wearable sensors. The versatility of the demonstrated device allows a broad range of complex sample processing and analysis operations for health monitoring applications.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1. Wearable device. a) On-body positioning. Illustration of b) sensing, c) valving, d) mixing, and e) pumping functionalities. f) Control diagram of the device. g) The wearable device and its connection with a PCB. h) Stacking of a tape-based microfluidic module.

FIG. 2. Simulation and experimental profiles of mixing functionality. a) Thermal simulation for mixing electrodes. b) Velocity simulation. c) Diagram of mixing experimental set-up. d) Calculated mixing index obtained from microscopy images. Normalized mixing index versus e) voltage, and f) flow rate profiles. g) On-body device test verification.

FIG. 3. Simulation and experimental profiles of pumping functionality with two different designs. Thermal simulation of a) parallel and b) orthogonal electrodes. Liquid velocity simulation of c) parallel and d) orthogonal electrodes. Microscopy images of bead tracking along with liquid flow in e) parallel and f) orthogonal electrodes. Velocity versus voltage profiles for g) parallel and h) orthogonal electrodes.

FIG. 4. Valving simulation and experimental results. a) Thermal simulation of a valve. b) Hydrogel reversibility in on-body experiment. c) Hydrogel shrinkage versus temperature profile, where insets are microscopy images of the valve corresponding to three temperatures. d) Hydrogel shrinkage percentage versus time profile.

FIG. 5. Sensing profiles and experiments on integration of sensing and valving. a) Amperometric profile of different concentrations of glucose spiked in artificial sweat. b) Calibration plot. c) Blood and sweat glucose concentration measurements before and after food in-take for three subjects. d) Real-time monitoring of current with different glucose concentrations spiked in artificial sweat injected by a valve switched on and off e) Step-by-step device operation modes.

### DETAILED DESCRIPTION

To realize a fully autonomous lab-on-the-body platform with diverse operations, in-situ biofluid actuation and compartmentalization should be employed in conjunction with sensing capabilities. Example operations resulting from such functionalities include: 1) periodic and continuous monitoring (where mixing of old and new samples should be prevented), 2) in-situ sample processing and purification (for enhanced sensitivity and selectivity), and 3) advanced wearable assays targeting low concentration analytes (where mass-transport constraints should be overcome to deliver target analytes to a transducer's surface).

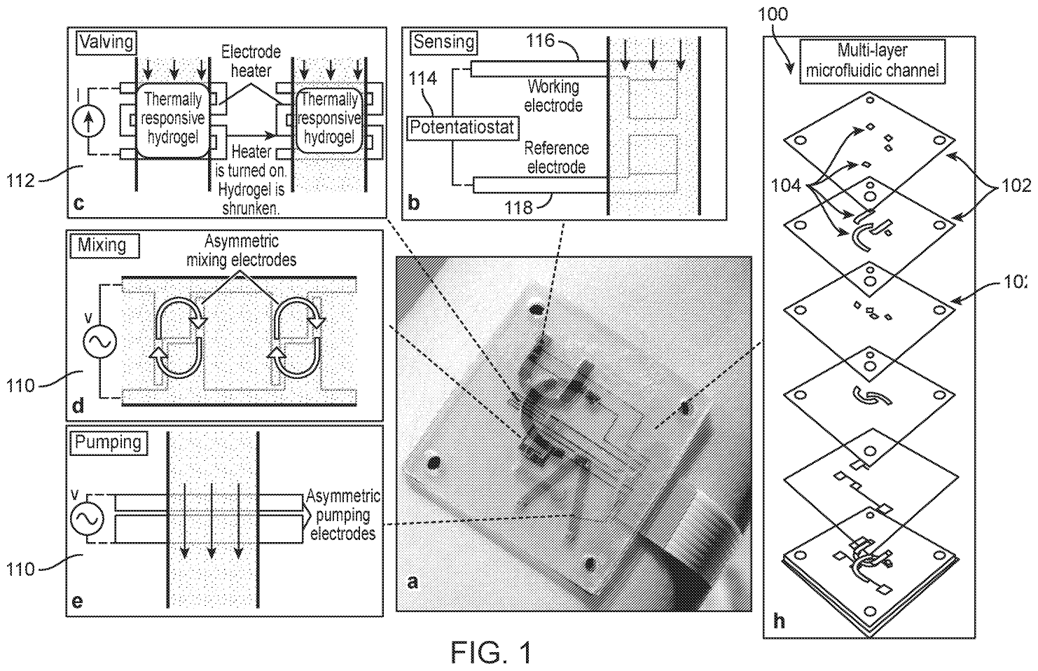

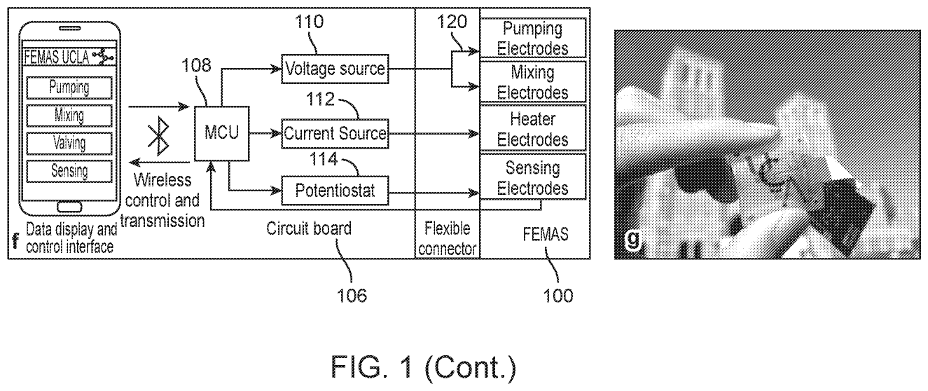

Here, in some embodiments, a suite of programmable electro-fluidic interfaces, integrated within a multi-layer flexible microfluidic module 100, are implemented to demonstrate biofluid actuation and control functionalities including pumping, mixing and valving for wearable sample analysis (FIG. 1). The microfluidic module 100 is formed by stacking multiple thin layers of adhesive polymeric substrates 102, each patterned with a functional electrode array (for one or more of biofluid actuation, valving, and sensing) and a set of pre-defined microfluidic conduits 104 to form channels, vias, and valves. The microfluidic conduits 104 of the stacked polymeric substrates 102 are interconnected to provide one or more respective multi-layer flow paths for biofluids across or through the multi-layer microfluidic module 100.

This mechanically flexible module 100, with adhesive contact, allows intimate and robust adherence to the human skin for extended wearability. The three-dimensional (3D) device architecture allows for the implementation of a diverse set of operations in a compact form. To achieve autonomous biofluid actuation, valving, and sensing with system-level operation, the microfluidic module 100 is interfaced with a miniaturized and wireless PCB 106 including, or to which is mounted, a controller 108 (or a microcontroller unit (MCU)) connected to and configured to direct operation of a voltage source 110, a current source 112, and a potentiostat 114. Data and control commands are bidirectionally relayed via wireless (e.g., Bluetooth) communication with a custom-developed mobile application.

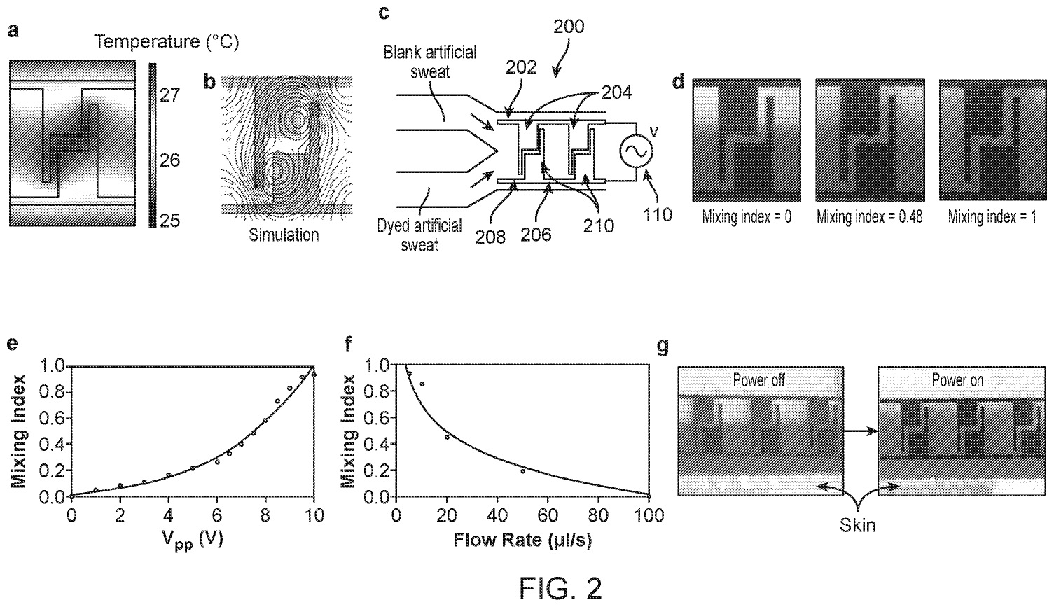

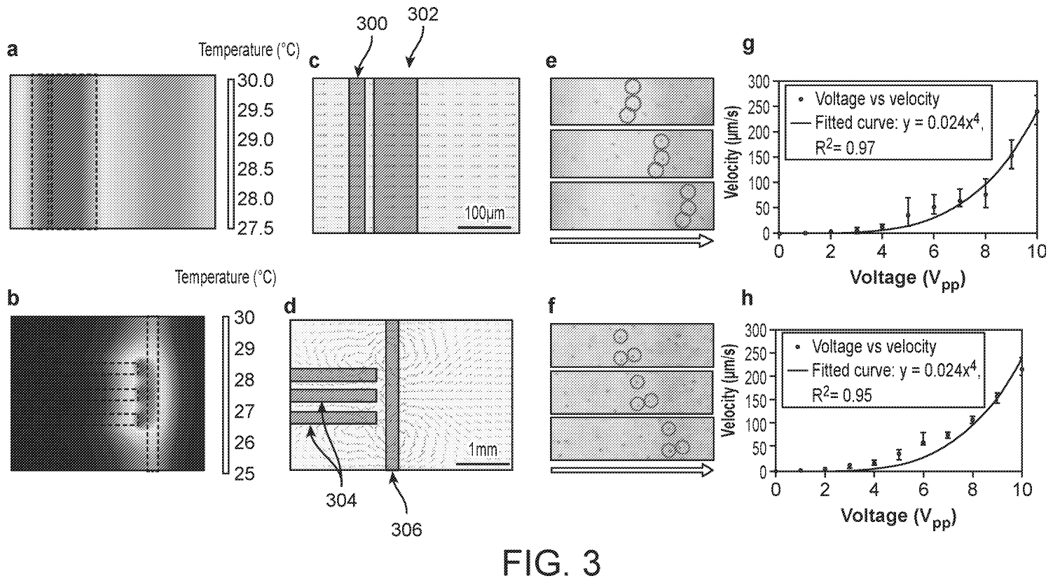

For electro-fluidic flow actuation, use is made of alternating current (AC) electrothermal flow (ACEF)-based phenomena, which are suitable for manipulation of microfluids with high conductivity (e.g., biofluids). This manner of actuation allows omission of bulky mechanical pumps, while allowing addressable, programmable and precise microfluid actuation through controlling applied voltage levels. ACEF arises in presence of a non-uniform electric field, which establishes temperature gradients and subsequently local permittivity and conductivity gradients within a fluid, leading to induced fluid motion. With proper symmetric and asymmetric design of electrode configurations, stirring and directional fluid motions are achieved to implement desired mixing and pumping functionalities, respectively (FIG. 1 in combination with FIGS. 2 and 3). Here, a rotationally symmetric mixing co-planar electrode pair is used to induce local in-plane micro-vortex flow profile (upon application of AC voltage, at an optimal frequency) (FIG. 2). The pair of mixing electrodes are configured in an interlocking manner, including a first electrode 200 including a first base member 202 and a set of first extending members 204 extending away from the first base member 202 toward a second electrode 206, which includes a second base member 208 and a set of second extending members 210 extending away from the second base member 208 toward the first electrode 200.

For pumping (FIG. 3), two configurations of asymmetric ACEF electrodes are used, in both of which the asymmetry in design is leveraged to create imbalanced temperature and electric fields in order to break symmetric competitive vortices, resulting in a net flow direction. One design includes a narrow electrode 300 (e.g., a width along a direction transverse to a lengthwise axis in a range of about 20 μm to about 60 μm, or about 40 μm) disposed adjacent to a wide electrode 302 (a width along a direction transverse to a lengthwise axis in a range of about 70 μm to about 110 μm, or about 90 μm, or, more generally, where the width of the wide electrode 302 is about 1.2 times or greater, about 1.5 times or greater, about 1.7 times or greater, or about 2 times or greater, and up to about 3 times or greater than the width of the narrow electrode 300), which are substantially parallel to one another (with respect to their lengthwise axes) and are separated by a distance in range of about 10 μm to about 50 μm, or about 30 μm, and patterned onto a bottom of a conduit to establish an intended non-uniform electric field profile. To validate the pumping functionality, about 1.4 V_{RMS} (>about 1 MHz) is applied across the asymmetric electrode pair 300 and 302, and the motion of the fluid is tracked, through time-sequential imaging of a group of microbeads suspended in artificial sweat (FIG. 3e). By increasing the applied voltage levels across the pumping electrode pair 300 and 302, validation is made that the induced velocity profile is correlated with the fourth power of the applied voltage (R²=0.97, FIG. 3g). Another design includes a set of first electrodes 304, which are substantially parallel to one another (with respect to their lengthwise axes), a second electrode 306, which is adjacent to and separated from the set of first electrodes 304, and is substantially perpendicular to the set of first electrodes 304 (with respect to their lengthwise axes), and where the set of first electrodes 304 and the second electrode 306 have substantially a same width. By increasing the applied voltage levels across the set of first electrodes 304 and the second electrode 306, validation is made that the induced velocity profile is correlated with the fourth power of the applied voltage (R²=0.95, FIG. 3h).

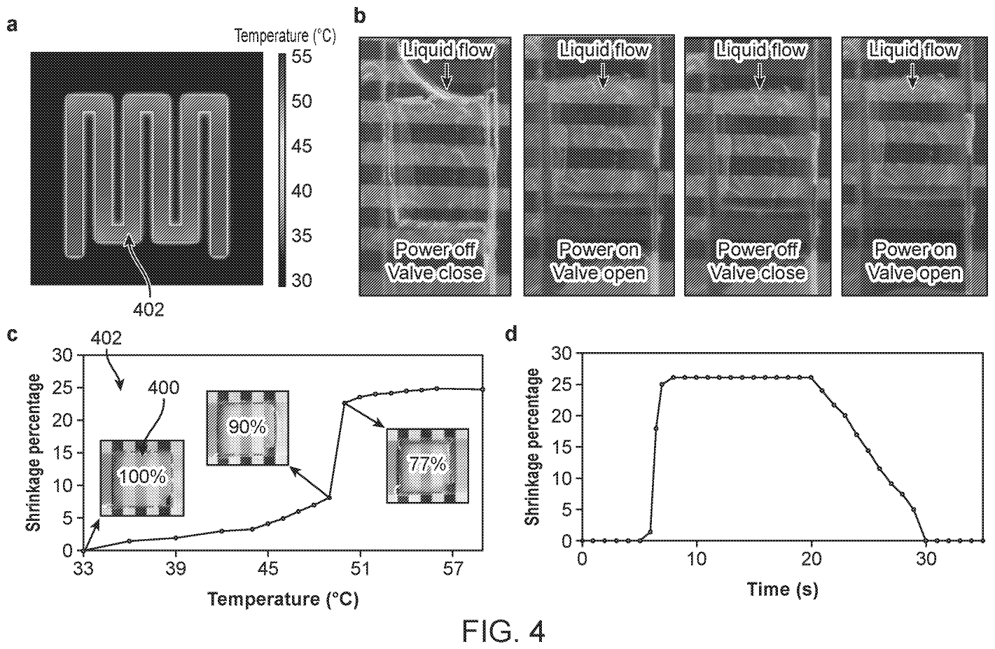

To realize biofluid compartmentalization, a wearable valve is devised, where microfluidic flow is permitted/blocked reversibly, through shrinkage/expansion of a hydrogel 400 (FIG. 4). In this way, the wearable valve overcomes constraints of comparative valves, which include bulky components and external control equipment, preventing their use for wearable applications. The valve is comprised of the thermal-stimuli-responsive hydrogel 400 (embedded in a microfluidic conduit) and a programmable heater electrode 402 (patterned on a bottom of the conduit). In some embodiments, the heater electrode 402 has an undulating or serpentine configuration, to enhance thermal stimuli applied through the heater electrode 402. In some embodiments, use is made of poly(N-isopropylacrylamide) (PNIPAM) as the hydrogel 400, which significantly shrinks in response to local temperature increments. The PNIPAM-valve is opened by activating the heater electrode 402 to heat the hydrogel 400 above its transition temperature, and reversibly, it can be closed by de-activating the heater electrode 402. In one demonstration, by applying a voltage (about 3 V) across terminals of the heater electrode 402, a local temperature is maintained above about 48° C., to subsequently shrink the hydrogel 400 and open the microfluidic conduit.

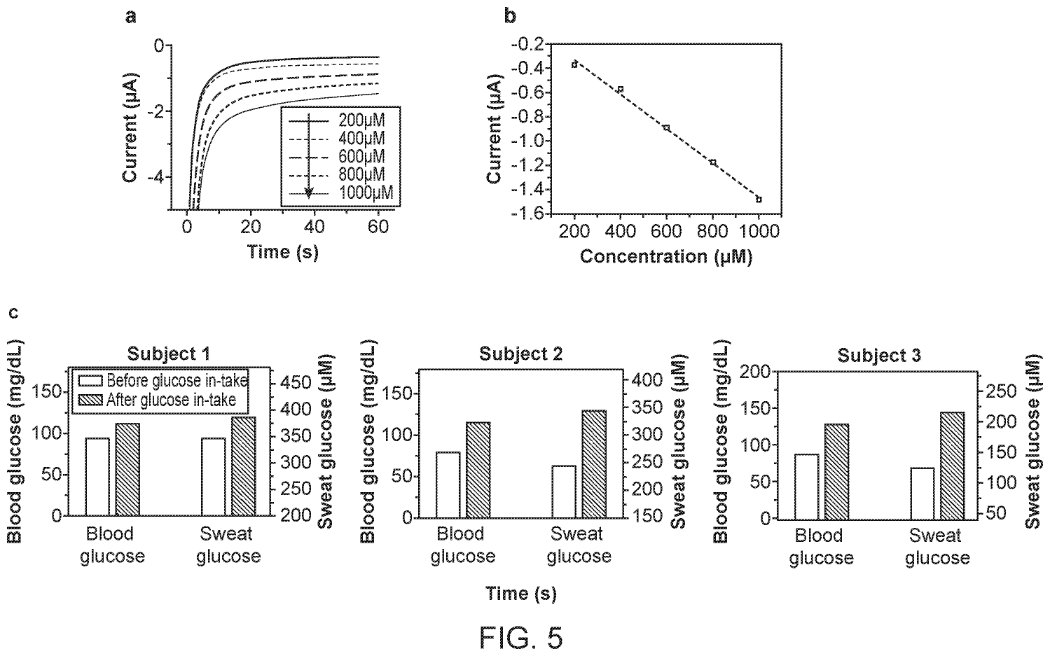

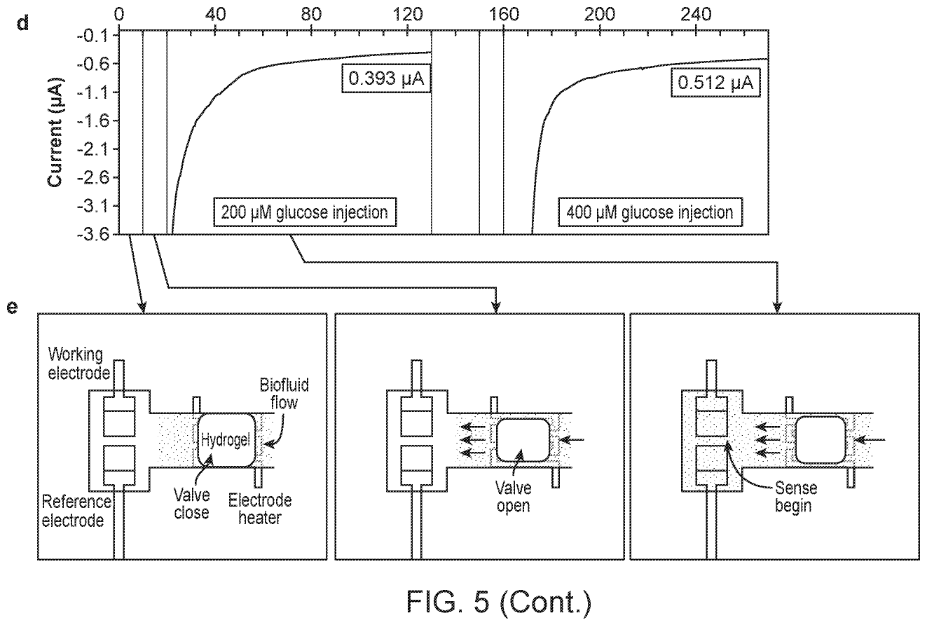

The versatility of the microfluidic module 100 allows for combining electro-fluidic actuation and sensing capabilities to realize a sample analysis system. To demonstrate the platform's biomarker sensing capability, an enzymatic sensing electrode pair is developed and embedded in the microfluidic module 100 (FIG. 1). The sensing electrode pair includes a working electrode 116, functionalized with a sensing layer including glucose oxidase enzymes entrapped in a chitosan film, and a Ag/AgCl electrode 118, which serves as both a reference and a counter electrode (FIG. 1). An amperometric interface outputs electrical current in correlation to a glucose concentration in a sample (FIG. 5a, b).

To achieve system-level operations with wireless control and data transmission (FIG. 1), the PCB 106 includes the controller 108 that can be programmed to control actuation circuitries and relay received data from a sensing circuitry via Bluetooth. The actuation circuitries include the AC voltage source 110, which is connected to and activates pumping and mixing electrodes, as well as the direct current (DC) source 112, which is connected to and activates heater electrodes. The sensing circuitry includes the potentiostat 114 (and a low-pass filter), which are connected to sensing electrodes for electrochemical sensor output acquisition and processing. The actuation and sensing circuitries on the PCB 106 are connected to the microfluidic module 100 through a flexible connector 120. The entire system-level device can be powered by a single miniaturized power source in the form of a rechargeable lithium-ion polymer battery with a nominal voltage of about 3.7 V.

Intended biofluid actuation, valving, and sensing operations are validated through a combination of simulation, in-vitro characterization, and on-body human subject testing. Specifically, the biofluid actuation and valving in-vitro characterization results are in agreement with electrothermal simulation and theoretically predicted trends, and the intended operations are validated through on-body experiments. To demonstrate the potential clinical application of the platform, demonstration is made of the elevation of glucose in iontophoretically-stimulated sweat after glucose intake in fasting subjects (FIG. 5c). The system-level operation is shown in the context of compartmentalized sample analysis of glucose (FIG. 5d, e). Overall, the results demonstrate the potential of the devised methodologies to perform complex sample processing and analysis operations, ultimately realizing a fully autonomous lab-on-the-body platform for a broad range of health care applications.

### Example Embodiments

The following are example embodiments of this disclosure.

### First Aspect

In some embodiments, a device for biofluid processing and analysis includes a microfluidic module including multiple stacked layers, each layer of the stacked layers defines a respective conduit, and conduits of the stacked layers are interconnected to provide a flow path for a biofluid.

In some embodiments, at least one layer of the stacked layers includes a polymeric substrate defining a respective conduit, and a set of pumping electrodes disposed along the conduit. In some embodiments, the set of pumping electrodes includes a first pumping electrode and a second pumping electrode spaced from the first pumping electrode and substantially parallel to the first pumping electrode, and a width of the first pumping electrode and a width of the second pumping electrode are different. In some embodiments, the set of pumping electrodes includes multiple first pumping electrodes, which are substantially parallel to one another, and a second pumping electrode that is substantially perpendicular to the first pumping electrodes. In any of the foregoing embodiments, the device further includes a voltage source connected to the set of pumping electrodes.

In some embodiments, at least one layer of the stacked layers includes a polymeric substrate defining a respective conduit, and a set of mixing electrodes disposed along the conduit. In some embodiments, the set of mixing electrodes includes a first mixing electrode and a second mixing electrode spaced from and interlocking with the first mixing electrode. In some embodiments, the first mixing electrode includes a first base member and a set of first extending members extending away from the first base member toward the second mixing electrode, and the second mixing electrode includes a second base member and a set of second extending members extending away from the second base member toward the first mixing electrode. In any of the foregoing embodiments, the device further includes a voltage source connected to the set of mixing electrodes.

In some embodiments, at least one layer of the stacked layers includes a polymeric substrate defining a respective conduit, a heater electrode disposed along the conduit, and a thermally responsive hydrogel disposed along the conduit and adjacent to the heater electrode. In any of the foregoing embodiments, the device further includes a current source connected to the heater electrode.

In some embodiments, at least one layer of the stacked layers includes a polymeric substrate defining a respective conduit, a working electrode disposed along the conduit and including a sensing layer, and a reference electrode disposed along the conduit and adjacent to the working electrode. In any of the foregoing embodiments, the device further includes a potentiostat connected to the working electrode and the reference electrode.

In some embodiments, the device further includes a controller connected to the voltage source, the current source, or the potentiostat.

### Second Aspect

In some embodiments, a device for biofluid processing and analysis includes a microfluidic module including multiple stacked layers, each layer of the stacked layers defines a respective conduit, conduits of the stacked layers are interconnected to provide a flow path for a biofluid, and the stacked layers include: a valve disposed along the flow path and including a heater electrode and a thermally responsive hydrogel disposed adjacent to the heater electrode; and a sensing electrode pair disposed along the flow path.

In some embodiments, the device further includes: a current source connected to the valve; a potentiostat connected to the sensing electrode pair; and a controller connected to the current source and the potentiostat to direct operation of the current source and the potentiostat.

In some embodiments, the sensing electrode pair is disposed downstream from the valve along the flow path. In some embodiments, the valve and the sensing electrode pair are disposed in different layers of the stacked layers. In some embodiments, the valve and the sensing electrode pair are disposed in a same layer of the stacked layers.

### Third Aspect

In some embodiments, a device for biofluid processing and analysis includes a microfluidic module including multiple stacked layers, each layer of the stacked layers defines a respective conduit, conduits of the stacked layers are interconnected to provide a flow path for a biofluid, and the stacked layers include: a set of pumping electrodes disposed along the flow path; and a sensing electrode pair disposed along the flow path.

In some embodiments, the set of pumping electrodes includes a first pumping electrode and a second pumping electrode spaced from the first pumping electrode and substantially parallel to the first pumping electrode, and a width of the first pumping electrode and a width of the second pumping electrode are different.

In some embodiments, the set of pumping electrodes includes multiple first pumping electrodes, which are substantially parallel to one another, and a second pumping electrode that is substantially perpendicular to the first pumping electrodes.

In some embodiments, the device further includes: a voltage source connected to the set of pumping electrodes; a potentiostat connected to the sensing electrode pair; and a controller connected to the voltage source and the potentiostat to direct operation of the voltage source and the potentiostat.

In some embodiments, the sensing electrode pair is disposed downstream from the set of pumping electrodes along the flow path. In some embodiments, the set of pumping electrodes and the sensing electrode pair are disposed in different layers of the stacked layers. In some embodiments, the set of pumping electrodes and the sensing electrode pair are disposed in a same layer of the stacked layers.

### Fourth Aspect

In some embodiments, a device for biofluid processing and analysis includes a microfluidic module including multiple stacked layers, each layer of the stacked layers defines a respective conduit, conduits of the stacked layers are interconnected to provide a flow path for a biofluid, and the stacked layers include: a set of mixing electrodes disposed along the flow path and including a first mixing electrode and a second mixing electrode spaced from and interlocking with the first mixing electrode; and a sensing electrode pair disposed along the flow path.

In some embodiments, the device further includes: a voltage source connected to the set of mixing electrodes; a potentiostat connected to the sensing electrode pair; and a controller connected to the voltage source and the potentiostat to direct operation of the voltage source and the potentiostat.

In some embodiments, the sensing electrode pair is disposed downstream from the set of mixing electrodes along the flow path. In some embodiments, the set of mixing electrodes and the sensing electrode pair are disposed in different layers of the stacked layers. In some embodiments, the set of mixing electrodes and the sensing electrode pair are disposed in a same layer of the stacked layers.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, a first numerical value can be “substantially” or “about” the same as a second numerical value if the first numerical value is within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, substantially parallel can refer to a range of angular variation relative to 0° of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°. For example, substantially perpendicular can refer to a range of angular variation relative to 90° of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

In the description of some embodiments, an object provided “on,” “over,” “on top of,” or “below” another object can encompass cases where the former object is directly adjoining (e.g., in physical or direct contact with) the latter object, as well as cases where one or more intervening objects are located between the former object and the latter object.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

Some embodiments of this disclosure relate to a non-transitory computer-readable storage medium having computer code or instructions thereon for performing various processor-implemented operations. The term “computer-readable storage medium” is used to include any medium that is capable of storing or encoding a sequence of instructions or computer code for performing the operations, methodologies, and techniques described herein. The media and computer code may be those specially designed and constructed for the purposes of the embodiments of the disclosure, or they may be of the kind available to those having skill in the computer software arts. Examples of computer-readable storage media include volatile and non-volatile memory for storing information. Examples of memory include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like. Examples of computer code include machine code, such as produced by a compiler, and files containing higher-level code that are executed by a processor using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computing device via a transmission channel. Another embodiment of the disclosure may be implemented in hardwired circuitry in place of, or in combination with, processor-executable software instructions.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

1. A device for biofluid processing and analysis, comprising:
a microfluidic module including a plurality of stacked layers, each layer of the stacked layers defines a respective conduit, and conduits of the stacked layers are interconnected to provide a flow path for a biofluid.

2. The device of claim 1, wherein at least one layer of the stacked layers includes a polymeric substrate defining a respective conduit, and a set of pumping electrodes disposed along the conduit.

3. The device of claim 2, wherein the set of pumping electrodes includes a first pumping electrode and a second pumping electrode spaced from the first pumping electrode and substantially parallel to the first pumping electrode, and a width of the first pumping electrode and a width of the second pumping electrode are different.

4. The device of claim 2, wherein the set of pumping electrodes includes a plurality of first pumping electrodes, which are substantially parallel to one another, and a second pumping electrode that is substantially perpendicular to the first pumping electrodes.

5. The device of claim 4, further comprising a voltage source connected to the set of pumping electrodes.

6. The device of claim 1, wherein at least one layer of the stacked layers includes a polymeric substrate defining a respective conduit, and a set of mixing electrodes disposed along the conduit.

7. The device of claim 6, wherein the set of mixing electrodes includes a first mixing electrode and a second mixing electrode spaced from and interlocking with the first mixing electrode.

8. The device of claim 7, wherein the first mixing electrode includes a first base member and a set of first extending members extending away from the first base member toward the second mixing electrode, and the second mixing electrode includes a second base member and a set of second extending members extending away from the second base member toward the first mixing electrode.

9. The device of claim 8, further comprising a voltage source connected to the set of mixing electrodes.

10. The device of claim 1, wherein at least one layer of the stacked layers includes a polymeric substrate defining a respective conduit, a heater electrode disposed along the conduit, and a thermally responsive hydrogel disposed along the conduit and adjacent to the heater electrode.

11. The device of claim 10, further comprising a current source connected to the heater electrode.

12. The device of claim 1, wherein at least one layer of the stacked layers includes a polymeric substrate defining a respective conduit, a working electrode disposed along the conduit and including a sensing layer, and a reference electrode disposed along the conduit and adjacent to the working electrode.

13. The device of claim 12, further comprising a potentiostat connected to the working electrode and the reference electrode.

14. The device of claim 13, further comprising a controller connected to the voltage source, the current source, or the potentiostat.

15. A device for biofluid processing and analysis, comprising:
a microfluidic module including a plurality of stacked layers, each layer of the stacked layers defines a respective conduit, conduits of the stacked layers are interconnected to provide a flow path for a biofluid, and the stacked layers including:
a valve disposed along the flow path and including a heater electrode and a thermally responsive hydrogel disposed adjacent to the heater electrode; and
a sensing electrode pair disposed along the flow path.

16. The device of claim 15, further comprising:
a current source connected to the valve;
a potentiostat connected to the sensing electrode pair; and
a controller connected to the current source and the potentiostat to direct operation of the current source and the potentiostat.

17. A device for biofluid processing and analysis, comprising:
a microfluidic module including a plurality of stacked layers, each layer of the stacked layers defines a respective conduit, conduits of the stacked layers are interconnected to provide a flow path for a biofluid, and the stacked layers including:
a set of pumping electrodes disposed along the flow path; and
a sensing electrode pair disposed along the flow path.

18. The device of claim 17, wherein:
the set of pumping electrodes includes a first pumping electrode and a second pumping electrode spaced from the first pumping electrode and substantially parallel to the first pumping electrode, and a width of the first pumping electrode and a width of the second pumping electrode are different; or
the set of pumping electrodes includes a plurality of first pumping electrodes, which are substantially parallel to one another, and a second pumping electrode that is substantially perpendicular to the first pumping electrodes.

19. The device of claim 18, further comprising:
a voltage source connected to the set of pumping electrodes;
a potentiostat connected to the sensing electrode pair; and
a controller connected to the voltage source and the potentiostat to direct operation of the voltage source and the potentiostat.

20. A device for biofluid processing and analysis, comprising:
a microfluidic module including a plurality of stacked layers, each layer of the stacked layers defines a respective conduit, conduits of the stacked layers are interconnected to provide a flow path for a biofluid, and the stacked layers including:
a set of mixing electrodes disposed along the flow path and including a first mixing electrode and a second mixing electrode spaced from and interlocking with the first mixing electrode; and
a sensing electrode pair disposed along the flow path.

21. The device of claim 20, further comprising:
a voltage source connected to the set of mixing electrodes;
a potentiostat connected to the sensing electrode pair; and
a controller connected to the voltage source and the potentiostat to direct operation of the voltage source and the potentiostat.
